(19) United States
(12) Patent Application Publication
(10) Pub. No.: US 2003/0186978 A1
(43) Pub. Date: Oct. 2, 2003

(54) Novel telomerase inhibitors

(76) Inventors: Alberto Bargiotti, Milano (IT); Antonella Ermoli, Buccinasco (IT); Maria Menichincheri, Milano (IT); Ermes Vanotti, Milano (IT); Luisella Bonomini, Cesano (IT); Antonella Fretta, Corsico (IT)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/311,179

(22) PCT Filed: Jun. 8, 2001
(86) PCT No.: PCT/EP01/06626

(30) Foreign Application Priority Data
Jun. 16, 2000 (GB) 0014861.9

(51) Int. Cl. A61K 31/5377; A61K 31/522; C07D 473/18

(52) U.S. Cl. 514/234.5; 544/276; 514/263.37; 514/263.38; 514/252.16; 514/263.2; 514/263.21; 514/263.22; 514/263.24; 544/118

(57) ABSTRACT

The present invention relates to novel telomerase inhibitors possessing antitumor activity and to a process for preparing the same. Furthermore, these compounds enhance the efficacy of other chemotherapeutic agents, in the treatment of cancer.

### BACKGROUND OF THE INVENTION

The present invention relates to new telomerase inhibitors possessing antitumor activity and, to a process for preparing the same.

Cancer is one of the major causes of disease despite the great effort and investments in research and development during the last decades. In addition to that, most cancer patients still die due to metastatic disease. In the same time, despite the great increase in the knowledge and understanding of the regulatory mechanisms involved in the onset of malignancy, currently available treatments (including surgery, radiation and a variety of cytoreductive and hormone-based drugs, used alone or in combination) are still highly non specific and toxic to the patient, causing severe side effects including nausea and vomiting, hair loss, diarrhea, fatigue, ulcerations and the like. These evidences indicate the need for new and more effective anti-cancer therapies. Recently an understanding of the mechanisms by which normal cells reach the state of senescence, i.e. the loss of proliferative capacity that cells normally undergo in the cellular aging process, has begun to emerge and in this respect telomerase appears to have a central role. Telomerase is a ribonucleoprotein enzyme responsible in most eukaryotes for the complete replication of chromosome ends, or telomeres, that are tandemly repeated DNA sequences (in particular human telomeres are formed by 5′-TTAGGG repeats). Telomerase synthesises one strand of the telomeric DNA using as a template a sequence contained within the RNA component of the enzyme necessary for the addition of the short sequence repeats (TTAGGG) to the chromosome 3′ end (see Blackburn 1992, Annu. Rev. Biochem., 61, 113-129). In most human somatic cells telomerase activity cannot be detected and telomeres shorten with successive cell division: in fact actively dividing normal cells have the potential to lose 50-200 base pairs after each round of cell division, due to the discontinuous synthesis of DNA lagging strand, finally resulting in shortening of telomeres. Recently scientists have hypothesised that the cumulative loss of telomeric DNA over repeated cell divisions may act as a trigger of cellular senescence and aging, and that regulation of telomerase may have important biological implications (see Harley 1991, Mutation Research, 256, 271-282). In fact in the absence of telomerase, telomeres shortening will eventually lead to cellular senescence by various mechanisms. This phenomenon, thought to be responsible for cellular aging, is termed the “mitotic clock” (Holt et al. Nat. Biotechnol., 1996, 15, 1734-1741). Conversely telomerase is restored in immortalised cell lines and in more than 85% of human tumors, thus maintaining telomere length constant (Shay, J. W. and Bacchetti, S. Eur. J. Cancer, 1997, 33, 787-791). Thus in cancer cells having telomerase activity and where the malignant phenotype is due to the loss of cell cycle or growth controls or other genetic damage, telomeric DNA is not lost during cell division, thereby allowing the cancer cells to become immortal, leading to a terminal prognosis for the patient. Actually it has been demonstrated that telomerase inhibition can lead to telomere shortening in tumors and senescent phenotype (Feng et al Science, 1995, 269, 1236-1241). Moreover it has been recently shown (Hahn et al. Nature Med., 1999, 5, 1164-1170) that inhibition of telomerase activity by expressing in tumor cells a catalytically-inactive form of human TERT (TElomerase Reverse Transcriptase, the catalytic subunit of the enzyme) can cause telomere shortening and arrest of cell growth. In addition peptide-nucleic acids and 2′-O-MeRNA oligomers complementary to the template region of the RNA component of the enzyme have been reported to cause inhibition of telomerase activity, telomere shortening and cell death in certain tumor cell lines (Herbert et al. PNAS, 1999, 96, 14276-14281; Shammas et al. Oncogene, 1999, 18, 6191-6200). These data strongly support inhibition of telomerase activity as an innovative, selective and useful method for the development of new anticancer agents. Thus compounds that inhibit telomerase activity can be used to treat cancer, as cancer cells express telomerase activity while normal human somatic cells do not express telomerase activity at biologically relevant levels (i.e., at levels sufficient to maintain telomere length over many cell divisions). In particular, the compounds of the present invention can provide a highly general method of treating many—if not most—malignancies, as demonstrated by the highly varied human tumor cell lines and tumors having telomerase activity. The compounds of the present invention are also expected to exhibit greater safety and to lack toxic effects in comparison with traditional chemotherapeutic anticancer agents, as they can be effective in providing treatments that discriminate between malignant and normal cells to a high degree, avoiding many of the deleterious side-effects present with most current chemotherapeutic regimes which rely on agents that kill dividing cells indiscriminately.

The present invention relates to novel purinic derivatives, to the use of them as therapeutic agents, in particular as antitumoral agents, to a process for their preparation and to pharmaceutical compositions comprising them. These and other aspects of the invention are described in greater detail below.

Object of the present invention are 3,7 disubstituted purines of formula (I)

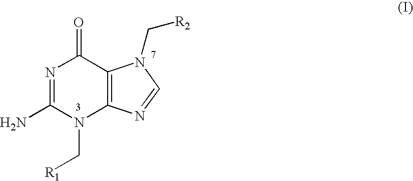

wherein

R₁ and R₂ represent each independently:

a) hydrogen;

b) phenyl unsubstituted or substituted by from 1 to 3 substituents chosen from a halogen, C₁-C₆ alkyl, C₁-C₆ alkoxy, hydroxy, carboxy, sulfo, cyano, nitro, amino, C₁-C₆ dialkylamino, a C₁-C₆ tetraalkylammonium halide, C₁-C₄ acylamino, (C₁-C₆ alkoxy)carbonyl, carbamoyl, (C₁-C₆ alkyl)carbamoyl, (C₁-C₆ dialkyl) carbamoyl, phenylcarbamoyl, guanidino, (C₁-C₆ alkyl) sulfonylamino, phenylsulfonylamino, (C₁-C₆ alkyl)aminosulfonyl, phenylaminosulfonyl, and C₅-C₇ cycloalkyl;

c) a group of formula

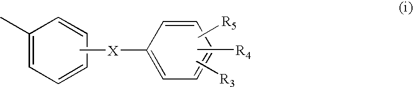

wherein

X is a bond, O or (CH₂)ₘ wherein m is an integer from 1 to 6;

R₃, R₄ and R₅ are, at the same time, hydrogen or R₃, R₄ and R₅ are chosen independently from hydrogen, a halogen, C₁-C₄ alkoxy, hydroxy, cyano, nitro, C₁-C₆ alkyl, halo C₁-C₆ alkyl, carboxy, sulfo, (C₁-C₆ alkoxy)carbonyl, amino and C₁-C₄ dialkylamino;

d) a monocyclic heteroaryl chosen from imidazolyl, pyrazolyl, oxadiazolyl, pyrrolyl, furanyl, thiadiazolyl, oxazolyl, thiazolyl, tetrazolyl, piperazinyl, N-alkyl piperazinyl, triazinyl, morpholinyl, pyridinyl, pyrimidinyl, pyrrolidinyl and piperidinyl;

e) a fused bicycle carbocyclic residue chosen from 1-naphthyl, 2-naphthyl and dihydronaphthalenyl;

f) a fused tricycle residue chosen from anthraquinonyl, phenothiazinyln, acridinyl and fluorenyl;

g) a fused benzoheterocyclic residue chosen from benzodioxinyl, benzodioxolyl, benzofuranyl, benzothiazolyl, a benzothiazolium halide, benzothiophenyl, benzoimidazolyl, a benzoimidazolium halide, benzoxazolyl, a benzoxazolium halide, benzoxadiazolyl, quinolinyl, isoquinolinyl and quinazolinyl;

h) a phenylheterocycle residue chosen from phenylimidazolyl, phenylpyrazolyl, phenyloxadiazolyl, phenylpyrrolyl, phenylfuranyl, phenylthiadiazolyl, phenyloxazolyl, phenylthiazolyl, phenyltetrazolyl, phenylpiperazinyl, phenyl-N-alkyl piperazinyl, phenyltriazinyl, phenylmorpholinyl, phenylpyridinyl, phenylpyrimidinyl, phenylpyrrolidinyl and phenylpiperidinyl; provided that R₁ and R₂ are not at the same time hydrogen, and when R₁ is hydrogen, R₂ is not unsubstituted phenyl, and the pharmaceutically acceptable salts thereof.

The present invention includes within its scope all possible isomers, stereoisomers and optical isomers and their mixtures, and the metabolites and the metabolic precursors or bioprecursors of the compounds of formula (I). The compounds of the invention can be represented also by the following tautomeric formulae (Ia) and (Ib)

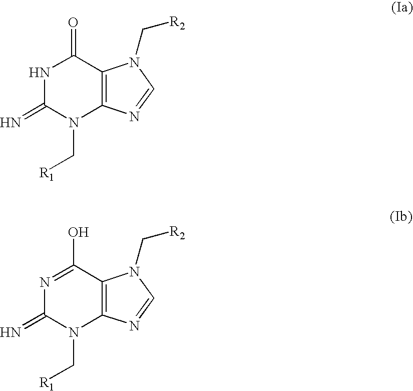

wherein R₁ and R₂ are as defined above. Accordingly, the chemical compounds provided by the present invention are named throughout the description of the invention according to the chemical nomenclature provided for the compounds of formula (I), (Ia) or (Ib) on the basis of the structural evidence validated by people skilled in the art. A halogen atom is chlorine, bromine, iodine or fluorine, preferably it is chlorine or fluorine. The alkyl and alkoxy groups may be branched or straight chain groups. A C₁-C₆ alkyl group is preferably a C₁-C₄ alkyl group, in particular a methyl or ethyl group. An alkoxy group is preferably a C₁-C₆ alkoxy group, more preferably a C₁-C₄ alkoxy group such as, e.g., methoxy, ethoxy, propoxy or butoxy. A (C₁-C₆ alkoxy)carbonyl group is preferably a (C₁-C₄ alkoxy)carbonyl group for example methoxycarbonyl, ethoxycarbonyl, propoxycarbonyl or butoxycarbonyl. A C₅-C₇ cycloalkyl group is cyclopentyl, cyclohexyl or cycloheptyl. A group of formula (i) as defined above is preferably a group of formula (ii) or (iii)

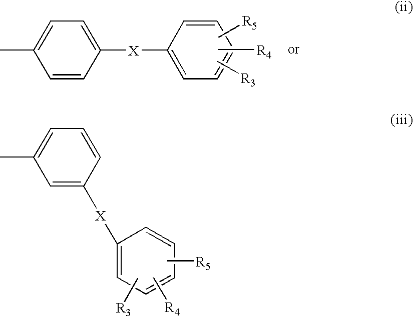

wherein X, R₃,R₄ and R₅ are as defined above. In a group of formula (i) or (ii) when X is (CH₂)ₘ, m is preferably an integer from 1 to 4.

Pharmaceutically acceptable salts of the compounds of the invention include acid addition salts, with inorganic, e.g. nitric, hydrochloric, hydrobromic, sulphuric, perchloric and phosphoric acids, or organic, e.g. acetic, propionic, glycolic, lactic, oxalic, malonic, malic, maleic, tartaric, citric, benzoic, cinnamic, mandelic and salicylic acids, and salts with inorganic, e.g. alkali metal, especially sodium or potassium bases, or alkaline-earth metal, especially calcium or magnesium bases, or with organic bases, e.g. alkylamines, preferably triethylamine.

As stated above, the present invention also includes within its scope pharmaceutically acceptable bio-precursors (otherwise known as pro-drugs) of the compounds of formula (I), i.e. compounds which have a different formula (I) above, but which nevertheless upon administration to a human being are converted directly or indirectly in vivo into a compound of formula (I).

Preferred compounds of the invention are compounds of formula (I) as defined above wherein, subjected to the above proviso, R₁ and R₂ represent each independently:

b′) phenyl unsubstituted or substituted by from 1 to 3 substituents chosen from a halogen, C₁-C₄ alkyl, C₁-C₄ alkoxy, hydroxy, carboxy, cyano, nitro, amino, and (C₁-C₄ alkoxy)carbonyl;

c′) a group of formula (ii) or (iii)

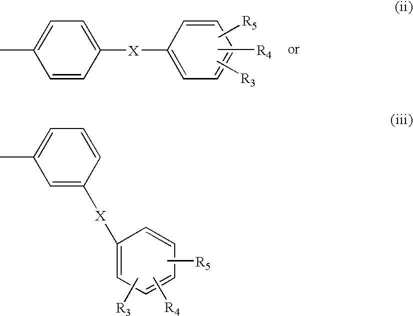

wherein

X is a bond, O or (CH₂)m wherein m is an integer from 1 to 4;

R₃, R₄ and R₅ are, at the same time, hydrogen or R₃, R₄ and R₅ are chosen independently from hydrogen, a halogen and haloCl-C₄ alkyl;

e′) a fused bicycle carbocyclic residue chosen from 1-naphthyl and 2-naphthyl;

f′) anthraquinonyl;

g′) a fused benzoheterocyclic residue chosen from quinolinyl, benzodioxolyl and benzoxadiazolyl;

h′) a phenyl heterocycle residue chosen from phenylimidazolyl, phenyltetrazolyl, phenylpyridyl and phenyl-N-alkyl-piperazinyl;

and the pharmaceutically acceptable salts thereof.

Examples of preferred compounds of the invention are:

2-amino-3,7-bis(2-naphthylmethyl)-3,7-dihydro-6H-purin-6-one (compound 1);

2-amino-3,7-bis-[(1,1′-biphenyl)-4-ylmethyl]-3,7-dihydro-6H-purin-6-one (compound 2);

2-amino-3,7-bis{[4′-(chloromethyl)[1,1′-biphenyl]-4-yl]methyl}-3,7-dihydro-6H-purin-6-one (compound 3);

2-amino-3,7-bis(3,4-dichlorobenzyl)-3,7-dihydro-6H-purin-6-one (compound 4);

2-amino-3-([1,1-biphenyl]-4-ylmethyl)-7-(2-naphthylmethyl)-3,7-dihydro-6H-purin-6-one (compound 5);

2-amino-3-(2-naphthylmethyl)-7-([1,1′-biphenyl]-4-ylmethyl)-3,7-dihydro-6H-purin-6-one (compound 6);

2-amino-3 (3,4-dichlorobenzyl)-7-(2-naphthylmethyl)-3,7-dihydro-6H-purin-6-one (compound 7);

2-amino-3-(3-phenoxybenzyl)-7-(2-naphthylmethyl)-3,7-dihydro-6H-purin-6-one (compound 8);

2-amino-3,7-bis (4-nitrobenzyl)-3,7-dihydro-6H-purin-6-one (compound 9);

2-amino-3,7-dibenzyl-3,7-dihydro-6H-purin-6-one (compound 10);

2-amino-3-(2-quinolinylmethyl)-7-(2-naphthylmethyl)-3,7-dihydro-6H-purin-6-one (compound 11);

2-amino-3-(2,1,3-benzoxadiazol-5-ylmethyl)-7-(2-naphthylmethyl)-3,7-dihydro-6H-purin-6-one (compound 12);

2-amino-3-(1,3-benzodioxol-5-ylmethyl)-7-(2-naphthylmethyl)-3,7-dihydro-6H-purin-6-one (compound 13);

2-amino-3-(4-nitrobenzyl)-7-(2-naphthylmethyl)-3,7-dihydro-6H-purin-6-one (compound 14);

2-amino-3-(4-aminobenzyl)-7-(2-naphthylmethyl)-3,7-dihydro-6H-purin-6-one (compound 15);

2-amino-3-(3,4-difluorobenzyl)-7-(2-naphthylmethyl)-3,7-dihydro-6H-purin-6-one (compound 16);

4-{[2-amino-7-(2-naphthylmethyl)-6-oxo-6,7-dihydro-3H-purin-3-yl]methyl}benzonitrile (compound 17);

2-amino-3-[4-(1H-imidazol-1-yl)benzyl]-7-(2-naphthylmethyl)-3,7-dihydro-6H-purin-6-one (compound 18);

2-{[2-amino-7-(2-naphthylmethyl)-6-oxo-6,7-dihydro-3H-purin-3-yl]methyl}anthra-9,10-quinone (compound 19);

methyl 4-{[2-amino-7-(2-naphthylmethyl)-6-oxo-6,7-dihydro-3H-purin-3-yl]methyl}benzoate (compound 20);

4-{[2-amino-7-(2-naphthylmethyl)-6-oxo-6,7-dihydro-3H-purin-3-yl]methyl}benzoic acid (compound 21);

2-amino-3-(3,4-dihydroxybenzyl)-7-(2-naphthylmethyl)-3,7-dihydro-6H-purin-6-one (compound 22);

2-amino-7-methyl-3-(2-naphthylmethyl)-3,7-dihydro-6H-purin-6-one (compound 23);

2-amino-3-methyl-7-(2-naphthylmethyl)-3,7-dihydro-6H-purin-6-one (compound 24);

2-amino-3-methyl-7-([1,1′-biphenyl]-4-ylmethyl)-3,7-dihydro-6H-purin-6-one (compound 25);

Methyl 4-{[2-amino-3-(2-naphthylmethyl)-6-oxo-3,6-dihydro-7H-purin-7-yl]methyl}benzoate (compound 26);

2-amino-7-(2-naphthylmethyl)-3-[4-(1H-tetraazol-5-yl)benzyl]-3,7-dihydro-6H-purin-6-one (compound 27);

Methyl 4-({2-amino-3-[4-(methoxycarbonyl)benzyl]-6-oxo-3,6-dihydro-7H-purin-7-yl}methyl) benzoate (compound 28);

Methyl 3-{[2-amino-7-(2-naphthylmethyl)-6-oxo-6,7-dihydro-3H-purin-3-yl]methyl}benzoate (compound 29);

3-{[2-amino-7-(2-naphthylmethyl)-6-oxo-6,7-dihydro-3H-purin-3-yl]methyl}benzoic acid (compound 30);

Methyl 2-{[2-amino-7-(2-naphthylmethyl)-6-oxo-6,7-dihydro-3H-purin-3-yl]methyl}benzoate (compound 31) and

4-{[2-amino-3-(2-naphthylmethyl)-6-oxo-3,6-dihydro-7H-purin-7-yl]methyl}benzoic acid (compound 32)

and the pharmaceutically acceptable salts thereof.

Another object of the present invention is a compound of formula (I) as defined above, for use as telomerase inhibitor.

A further object of the present invention is a compound of formula (I) as defined above, for use as a medicament, in particular as an antitumor agent.

A still another object of the present invention is a compound of formula (I) as defined above for use in treating a telomerase-modulated disease. In particular a compound of formula (I) according to the invention can be used for treating a cancer disease related to a deranged cancer cell growth mediated by telomerase enzyme activity.

A still further object of the present invention is to provide a pharmaceutical composition comprising a pharmaceutically acceptable carrier and/or diluent and, as an active principle, a compound of formula (I) as defined above.

The present invention also provides the use of a compound of formula (I) as defined above, in the preparation of a medicament for use as antitumor agent.

A method for inhibiting telomerase by using a compound of formula (I) as defined above is also an object of the invention.

According to the present invention, there is provided a process for the preparation of 3,7 disubstituted purines of formula (I) as defined above. In one embodiment of the process, a compound of formula (I) wherein R₁═R₂ are as defined above, can be prepared by a process comprising: the reaction of a compound of formula (IV)

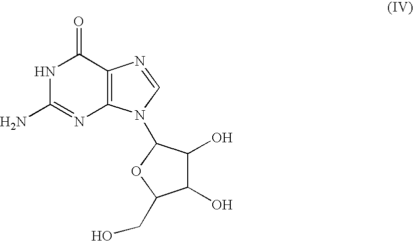

with a compound of formula (III)

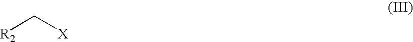

wherein

X is a suitable leaving group and R₂ is as defined above; and optionally salifying a compound of formula (I) as defined above, so obtaining a compound of formula (I) in the form of a pharmaceutically acceptable salt. A suitable leaving group is, e.g., a halogen, preferably Cl, Br or I, tosylate, mesylate or triflate. The reaction between a compound of formula (IV) as defined above and a compound of formula (III) as defined above, may be carried out, for example, in a suitable organic solvent such as, e.g., N,N-dimethylacetamide, dimethylformamide (DMF), tetrahydrofuran, dioxane, dimethoxyethane or toluene, at a temperature varying between about 60° C. and about 120° C., for a time of about 1 hour to about 15 hours, following, for example, literature methods as reported in J.Med.Chem. 1980, 357. Compounds of formula (III) and formula (IV) are known compounds or can be prepared by known methods. For example, a compound of formula (IV) wherein R₂ is the natural product guanosine and a compound of formula (III) can be prepared following methods well known in the art. For example, a compound of formula (III) can be prepared by halogenation reaction, with many methods known to people skilled in the art, of the corresponding alcohols that are commercially available, or alternatively can be prepared, for example, from the corresponding commercial esters, by standard methods. Examples of compounds prepared accordingly to this procedure are compounds 1-4, 9 and 10.

According to a preferred embodiment of the invention, a compound of formula (I), wherein R₁═R₂ or, more preferably, wherein R₁ is different from R₂, can be prepared by a process comprising: the reaction of a compound of formula (IV) as defined above, with a compound of formula (III) as defined above, to obtain a compound of formula (V)

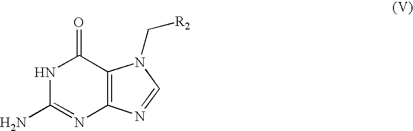

that can be represented also by the following tautomeric formulae (Va), (Vb), (Vc) and (Vd)

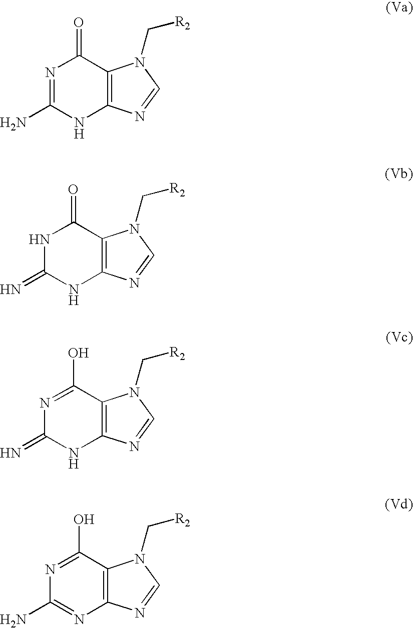

wherein R₂ is as defined above; the reaction of a compound of formula (V) with a compound of formula (VI)

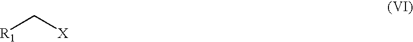

wherein

X is a suitable leaving group and R₁═R₂ or, more preferably, wherein R₁ is different from R₂ as defined above, and optionally salifying a compound of formula (I) as defined above, so obtaining a compound of formula (I) in the form of a pharmaceutically acceptable salt. A suitable leaving group is, e.g., a halogen, preferably Cl, Br or I, tosylate, mesylate, trifluoroacetate and triflate. When the reaction between the a compound of formula (IV) as defined above and a compound of formula (III) as defined above is performed in dimethylsulfoxide (DMSO) or DMF, at a temperature varying between about 20° C. and about 60° C., for a time of from about 1 hour to about 6 hours, as described, for example, in J. Org. Chem. 1986, 4180 or Synth. Comm. 1990, 2459, instead of a compound of formula (I) wherein R₁═R₂, a monosubstituted derivative of formula (V) as defined above can be obtained and used in the preparation of both symmetrical (where R₁═R₂) and unsymmetrical compounds of formula I (where R₁ is different from R₂) as described above. The reaction between a compound of formula (V), (Va), (Vb), (Vc) or (Vd) and a compound of formula (VI), may be carried out, foe example, in a suitable organic solvent such as DMF, N,N-dimethylacetamide, dimethylsulfoxide, tetrahydrofuran, dioxane or dimethoxyethane, optionally in the presence of both an inorganic base such as sodium or potassium hydride, sodium, potassium or barium hydroxide, sodium or potassium carbonate, or an organic base such as, for instance, potassium terbutoxide, methyllithium, butyllithium, lithiumdiisopropylamine, lithium, sodium or potassium hexamethyldisilazide, at a temperature varying between room temperature and about 120° C., for a time of about 1 hour to about 15 hours, following, for example, literature methods as reported in Synth. Comm. 1990, 2459. Examples of compounds prepared accordingly to this procedure are compounds 1-23 and 26-32.

According to a further a preferred embodiment of the invention, a compound of formula (I) wherein R₁═H can be prepared by a process comprising:

the reaction of a compound of formula (II)

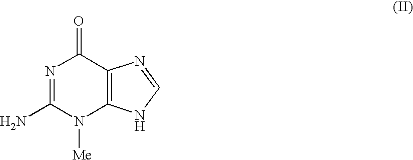

with a compound of formula (III)

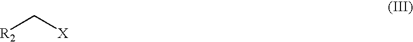

wherein X is a suitable leaving group and R₂ is as defined above, and optionally salifying a compound of formula (I) as defined above, so obtaining a compound of formula (I) in the form of a pharmaceutically acceptable salt. A suitable leaving group is, e.g., a halogen, preferably Cl, Br or I, tosylate, mesylate, trifluoroacetate and triflate. The reaction between a compound of formula (II) as defined above and a compound of formula (III) as defined above, may be carried out, for example in a suitable organic solvent such as, e.g., N,N-dimethylformamide, dimethylsulfoxide, N,N-dimethylacetamide, tetrahydrofuran, dioxane, dimethoxyethane or toluene, at a temperature varying between room temperature and about 120° C., for a time of about 1 hour to about 15 hours, optionally in the presence of both an inorganic base such as, e.g., sodium or potassium hydride, sodium, potassium or barium hydroxide, sodium or potassium carbonate or an organic base such as, for instance, potassium terbutoxide, methyllithium, butyllithium, lithiumdiisopropylamine, lithium, sodium or potassium hexamethyldisilazide, following, for example, literature methods as reported in Chem.Pharm. Bull. 1989, 37, 284. A compound of formula (II) is a commercially available compound. Examples of compounds prepared accordingly to this procedure are compounds 24 and 25.

The compounds of formula (I), (Ia) and (Ib) are herein defined as the “compounds of the present invention”, the “compounds of the invention” and/or the “active principles of the pharmaceutical compositions of the invention”.

The compounds of the invention can be administered in a variety of dosage forms, e.g. orally, in the form of tablets, capsules, lozengers, liquid solutions or suspensions; rectally, in the form of suppositories; parenterally, e.g. intramuscularly, intravenously, intradermally or subcuteneously; or topically. The dosage depends upon, for example, the compound of the invention employed, the age, weight, condition of the patient and administration route; specific dosage regimens may be fit to any particular subject on the basis of the individual need and the professional judgement of the person administering or supervising the administration of the aforesaid compounds. For example, the dosage adopted for the administration to adult humans may range from 0.001 to 100 mg of compound of the invention per kg of body weight; a particularly preferred range may be from 0.1 to 10 mg of compound of the invention per kg of body weight. The dosages may be administered at once or may be divided into a number of smaller doses to be administered at varying intervals of time. Pharmaceutical compositions containing, as an active ingredient, a compound of formula (I) or a pharmaceutically acceptable salt thereof in association with a pharmaceutically acceptable carrier and/or diluent, are also within the scope of the present invention.

These pharmaceutical compositions contain an amount of active ingredient, which is therapeutically effective to display antileukemic and/or antitumor activity. There may also be included as a part of the pharmaceutical compositions according to the invention, pharmaceutically acceptable binding agents and/or adjuvant materials. The active ingredients may also be mixed with other active principles, which do not impair the desired action and/or supplement the desired action. The pharmaceutical compositions containing the compounds of the invention are usually prepared following conventional methods and may be administered in a pharmaceutically suitable form. For example, the solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents, e.g. starches, arabic gums, gelatin, methylcellulose, microcrystalline cellulose, carboxymethylcellulose or polyvinyl pyrrolidone; diaggregating agents, e.g. a starch, alginic acid, alginates or sodium starch glycolate; effervescing mixtures; dyestuffs; sweetening agents, e.g. sucrose or saccharin; flavouring agents, e.g. peppermint, methylsalicylate or orange flavouring; wetting agents, such as lecithin, polysorbates, laurylsulphates; and, in general, non-toxic and pharmacologically inactive substances used in pharmaceutical formulations. When the dosage unit form is a capsule, it may contain, in addition to material of the above type, a liquid carrier such as, e.g., a fatty oil.

Said pharmaceutical preparations may be manufactured in known manner, for example, by means of mixing, granulating, tabletting, sugar-coating or film-coating processes. The liquid dispersions for oral administration may be, e.g. syrups, emulsions and suspensions. The syrups may contain as carrier, for example, saccharose or saccharose with glycerine and/or mannitol and/or sorbitol; in particular, a syrup to be administered to diabetic patients can contain as carriers only products not metabolizable to glucose, or metabolizable in very small amount to glucose, for example sorbitol. The suspensions and the emulsions may contain as carrier, for example, a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and, if desired, a suitable amount of lidocaine hydrochloride. The solutions for intravenous injections or infusions may contain as carrier, for example, sterile water, or preferably they may be in the form of sterile, aqueous, isotonic saline solution. The solutions or suspensions for parenteral therapeutic administration may also contain antibacterial agents, such as benzyl alcohol or methyl parabens; antioxidants, such as ascorbic acid or sodium bisulphite; chelating agents, such as ethylenediaminetetraacetic acid; buffers, such as acetates, citrates or phosphates and agents for the adjustment of tonicity, such as sodium chloride or dextrose. The parenteral preparation can be enclosed in ampoules, disposable syringes or multiple dose vials made of glass or plastic. The suppositories may contain together with the active compound a pharmaceutically acceptable carrier, e.g., coca-butter, polyethylene glycol, a polyoxyethylene sorbitan fatty acid ester surfactant or lecithin. Compositions for topical application, such as, e.g., creams, lotions or pastes, may be, e.g., prepared by admixing the active ingredient with a conventional oleaginous or emulsifying excipient.

Biological Activity

The telomerase activity of the compounds of the invention has been evaluated using a Flash Plate-based assay. The method proved to be sensitive, accurate and able to reproducibly identify compounds that inhibit telomerase activity in a dose-dependent manner. Other methods for determining the inhibitory concentration of a compound of the invention against telomerase can be employed as will be apparent to a person skilled in the art based on the disclosure herein.

Briefly, the assay mixture is costituted by:

telomerase enzyme diluted in a buffer, the composition of which has been selected to maintain the enzyme activity stable along the duration of the assay.

dNTPs, deoxynucleotides 5′-triphosphate.

biotynilated oligo as primer.

increasing concentrations of test compounds/positive control.

After two hours of incubation at 37° degrees the telomeric repeats added are evaluated by hybridization in solution with a 3′-labeled short oligonucleotide probe. The extent of hybridization is then quantitated by transferring the reaction mixture in a streptavidin-coated flash plate, where the binding between biotin and streptavidin occurs. The telomerase activity is proportional to the radioactivity measured and the inhibitory activity of the compounds is evaluated as IC₅₀ using the Sigma Plot fit program. With the above-described method, IC₅₀ values of the compounds of the present invention were determined. The results relative to a representative selection of compounds of the invention are shown in Table 1 below. The data reported in Table 1 are only indicative since obtained during the screening by fitting a limited number of experimental points.

TABLE 1

| Compound | IC₅₀(μM) |
|---|---|
| 1 | 4 |
| 2 | 3 |
| 3 | 5 |
| 4 | 4 |
| 5 | 2 |
| 6 | 2 |
| 7 | 3 |
| 8 | 3 |
| 19 | 4 |
| 27 | 19 |
| 29 | 21 |

The data reported in Table 1 clearly show the activity of the compounds according to the invention as telomerase inhibitors. A human or animal body may thus be treated by a method, which comprises the administration thereto of a pharmaceutically effective amount of a compound of formula (I) or a salt thereof. The condition of the human or animal can thereby be improved.

Combination chemotherapy using two or more anti-cancer drugs to treat malignant tumors in humans is currently in use in research and in the clinic. With regard to cancer the term “treating” or “treat” simply means that life expectancy of an individual affectd with a cancer will be increased, that one or more of the symptoms of the disease will be reduced and/or the quality of life will be enhanced.

The anti-cancer drugs may be, for example, topoisomerase inhibitors, antimetabolites, alkylating agents, antibiotics, antimicrotubule agents or anti-angiogenesis agents. Combinations of drugs are administered in an attempt to obtain a synergistic effect on most cancers, e.g., carcinomas, melanomas, lymphomas and sarcomas, and to reduce or eliminate emergence of drug-resistant cells and to reduce side effects to each drug. It is therefore a still further aspect of the present invention a combination therapy of a compound according to the invention with at least one other anti-cancer agent. The use of active subtances together provides improved therapeutic effect than employing the single agents alone. Antineoplastic agents suitable for combination with the compounds of the present invention include, but are not limited to:

topoisomerase I inhibitors such as camptothecins including irinotecan, SN-38, topotecan, 9-amino-camptothecin, 10,11-Methylenedioxy camptothecin and 9-nitro-camptothecin (rubitecan);

alkylating agents including nitrogen mustards such as, e.g., mechlorethamine, chlorambucil, melphalan, uracil mustard and estramustine; alkylsulfonates such as, e.g., busulfan improsulfan and piposulfan; oxazaphosphorines such as e.g., ifosfamide, cyclophosphamide, perfosfamide, and trophosphamide; and nitrosoureas such as, e.g., carmustine, lomustine and streptozocin;

antimicrotubule agents including taxanes such as , e.g., paclitaxel and docetaxel; and vinca alkaloids such as, e.g., vincristine, vinblastine, vinorelbine and vindesine,

antimetabolites including purines such as , e.g., 6-mercaptopurine, thioguanine, azathioprine, allopurinol, cladribine, fludarabine, pentostatin, and 2-chloro adenosine; fluoropyrimidines such as, e.g., 5-FU, fluorodeoxyuridine, ftorafur, 51′-deoxyfluorouridine, UFT, S-1 and capecitabine; and pyrimidine nucleosides such as, e.g., deoxycytidine, cytosine arabinoside, 5-azacytosine, gemcitabine, and 5-azacytosine-arabinoside;

hormones, hormonal analogues and hormonal antagonists including diethylstilbestrol, tamoxifen, exemestane, toremefine, tolmudex, flutamide, finasteride, stradiol, droloxifene, and medroxyprogesterone acetate; and

antibiotics including anthracyclines/anthracenediones such as, e.g., doxorubicin, daunorubicin, epirubicin, idarubicin and mitoxantrone.

A furhter class of compounds suitable for combination with the compounds of the present invention are antiangiogenic agents. More than 20 years ago, Folkman (Folkman J: Tumor angiogenesis: Therapeutic implications. N Engl J Med 285:1182-1186, 1971) proposed the hypothesis that solid tumor growth was dependent on the development of tumor-associated blood vessels, a process called angiogenesis. Numerous studies of experimental and human tumors have confirmed the central role of angiogenesis in solid tumor progression. Over the past decade it has become clear that inhibition of tumour angiogenesis is an effective anticancer treatment. It is therefore another object of the present invention a combination of a compound of the present invention with an antiangiogenesis agent.

Examples of agents with antiangiogenic activity include: SU 5416, AGM 1470 (TNP-470), a synthetic analogue of fumagillin a naturally secreted product of the fungus Aspergillus fumigates fresenius; angiostatin, a 38 kDa fragment of plasminogen; platelet factor 4 (endostatin); thalidomide; linomide; marimastat (BB-2516) and batimastat (BB-94).

Chemistry

The following examples illustrate but do not limit the invention.

### EXAMPLE 1

Compound 1

A mixture of guanosine hydrate (2.83 g; 10 mmols) and 2-naphthylmethyl bromide (4.41 g; 20 mmols) in N,N-dimethylacetamide (100 mL) is stirred at 90° C. for 3 hours. After solvent evaporation under reduced pressure the crude reaction product is purified by flash chromatography (eluant: dichloromethane/methanol 10:1) to yield 2-amino-3,7-bis(2-naphthylmethyl)-3,7-dihydro-6H-purin-6-one as a white solid. Yield: 45%.

¹H-NMR (400 Mhz, DMSOd₆), ppm: 5.5 (2H, s), 5.7 (2H, s), 7.0 (2H, s), 7.4 (1H, dd, J=1.5, 8.6Hz), 7.4-7.5 (4H, m), 7.5 (1H, dd, J=1.9, 8.6Hz), 7.7 (1H, s), 7.8-7.9 (7H, m), 8.1 (1H, s).

By analogous procedure the following compounds were prepared:

²-amino-3,7-bis(1,1′-biphenyl-4-ylmethyl)-3,7-dihydro-6H-purin-6-one (compound 2); ¹H-NMR (400 Mhz, DMSOd₆), ppm: 5.4 (2H, s), 5.6 (2H, s), 7.1 (2H, s), 7.2-7.6 (18H, m), 8.1 (1H, s).

2-amino-3,7-bis{[4′-(chloromethyl)[1,1′-biphenyl]-4-yl]methyl}-3,7-dihydro-6H-purin-6-one (compound 3); ¹H-NMR (400 Mhz, DMSOd₆), ppm: 4.75 (4H, s) , 5.35 (2H, s), 5.55 (2H, s), 7.0 (2H, s), 7.25-7.7 (16H, m), 8.1 (1H, s).

2-amino-3,7-bis(3,4-dichlorobenzyl)-3,7-dihydro-6H-purin-6-one (compound 4); ¹H-NMR (400 Mhz, DMSOd₆), ppm: 5.3 (2H, s), 5.5 (2H, s), 7.0 (2H, s), 7.1-7.7 (6H, m), 8.1 (1H, s).

2-amino-3,7-bis (4-nitrobenzyl)-3,7-dihydro-6H-purin-6-one (compound 9); ¹H-NMR (400 Mhz, DMSOd₆), ppm: 5.4 (2H, s), 5.6 (2H, s), 7.1 (2H, s), 7.4-7.6 (4H, 2d), 8.1 (1H, s); 8.2 (4H, m).

2-amino-3,7-dibenzyl-3,7-dihydro-6H-purin-6-one (compound 10); 1H-NMR (400 Mhz, DMSOd₆), ppm: 5.3 (2H, s), 5.5 (2H, s), 6.9 (2H, s), 7.2-7.4 (10H, m), 8.0 (1H, s).

### EXAMPLE 2

Compound 1

To a solution of guanosine hydrate (570 mg; 2 mmol) in anhydrous DMSO (3 mL) under argon 2-naphthylmethyl bromide (1.075 g; 4.8 mmol) is added and the reaction mixture stirred at room temperature for 4 h. Concentrated aq. HCl (1.5 mL) is added, the mixture is stirred for 0.5 h then poured into methanol (20 mL). The solution is concentrated and added of dichloromethane/methanol 2:1 (5 mL). The white precipitate is filtered and washed with dichloromethane. In this way 7-(2-naphthylmethyl) guanine hydrochloride (90% yield) is obtained and used for the second step. 7-(2-naphthylmethyl) guanine hydrochloride (20 mg; 0.62 mmol) and 2-naphthylmethyl bromide (144 mg; 0.65 mmol) are suspended in N,N-dimethylacetamide (15 mL) and the mixture is stirred at 90° C. for 3 h: The solvent is evaporated under reduced pressure and the crude reaction product is purified by flash chromatography (eluant dichloromethane/methanol 10:1) to yield 2-amino-3,7-bis(2-naphthylmethyl)-3,7-dihydro-6H-purin-6-one as a white solid. Yield: 42%. In the same way 2-amino-7-([1,1′-biphenyl]-4-ylmethyl)-1,7-dihydro-6H-purin-6-one hydrochloride and methyl 4-[(2-amino-6-oxo-1,6-dihydro-7H-purin-7-yl)methyl]benzoate hydrochloride have been prepared.

### EXAMPLE 3

Compound 5

7-(2-naphthylmethyl) guanine hydrochloride (200 mg; 0.62 mmol) and (1,1′-biphenyl)-4-ylmethyl chloride (132 mg; 0.65 mmol) are suspended in N,N-dimethylacetamide (15 mL) and the mixture is stirred at 120° C. for 8 h. The solvent is evaporated under reduced pressure and the crude reaction product is purified by flash chromatography (eluant dichloromethane/methanol 10:1) to yield 2-amino-3-(1,1′-biphenyl)-4-ylmethyl-7-(2-naphthylmethyl)-3,7-dihydro-6H-purin-6-one as a white solid. Yield: 46%. ¹H-NMR (400 Mhz, DMSOd₆), ppm: 5.4 (2H, s), 5.7 (2H, s), 7.0 (2H, s), 7.2-7.9 (16H, m), 8.1 (1H, s)

### EXAMPLE 4

Compound 1

To a suspension of 7-(2-naphthylmethyl) guanine (165 mg; 0.5 mmol) in anhydrous DMF (4 mL), NaH (1.2 mmol) is added and the mixture is stirred at room temperature for 2 h. A solution of 2-naphthylmethyl bromide (125 mg; 0.56 mmols) in anh. DMF (1 mL) is added and the reaction mixture is stirred for 3 h at room temperature. After solvent evaporation under reduced pressure the crude reaction product is purified by flash chromatography (eluant dichloromethane/methanol 20:1) to yield 2-amino-3,7-bis(2-naphthylmethyl)-3,7-dihydro-6H-purin-6-one as a white solid. Yield: 62%.

By analogous procedures, using the appropriate halide and the proper 7-substituted guanine, all the symmetrical (R₁═R₂) and unsymmetrical (R₁‡R₂) compounds of the patent can be synthesized and in particular:

starting from 7-(4-phenyl)benzyl guanine, 2-amino-3-(2-naphthylmethyl)-7-([1,1′-biphenyl]-4-ylmethyl)-3,7-dihydro-6H-purin-6-one (compound 6) was obtained. ¹H-NMR (400 Mhz, DMSOd₆), ppm: 5.5 (2H, s), 5.6 (2H, s), 7.0 (2H, s), 7.2-7.9 (16H, m), 8.1 (1H, s)

2-amino-3 (3,4-dichlorobenzyl)-7-(2-naphthylmethyl)-3,7-dihydro-6H-purin-6-one (compound 7). ¹H-NMR (400 Mhz, DMSOd₆), ppm: 5.3 (2H, s), 5.7 (2H, s), 7.0 (2H, s), 7.1-7.9 (10H, m), 8.1 (1H, s).

2-amino-7-(2-naphthylmethyl)-3-(3-phenoxybenzyl)-3,7-dihydro-6H-purin-6-one (compound 8). ¹H-NMR (400 Mhz, DMSOd₆), ppm: 5.3 (2H, s), 5.7 (2H, s), 6.8-7.9 (18H, m), 8.1 (1H, s).

2-amino-7-(2-naphthylmethyl)-3-(2-quinolinylmethyl)-3,7-dihydro-6H-purin-6-one (compound 11). 1H-NMR (400 Mhz, DMSOd₆), ppm: 5.6 (2H, s), 5.7 (2H, s), 7.0 (2H, s), 7.3 (1H, d), 7.4-7.9 (11H, m), 8.1 (1H, s) , 8.3 (1H, d).

2-amino-3-(2,1,3-benzoxadiazol-5-ylmethyl)-7-(2-naphthylmethyl)-3,7-dihydro-6H-purin-6-one (compound 12). ¹H-NMR (400 Mhz, DMSOd₆), ppm: 5.4 (2H, s), 5.7 (2H, s), 7.1 (2H, s), 7.4-8.0 (10H, m), 8.1 (1H, s).

2-amino-3-(1,3-benzodioxol-5-ylmethyl)-7-(2-naphthylmethyl)-3,7-dihydro-6H-purin-6-one (compound 13). ¹H-NMR (400 Mhz, DMSOd₆), ppm: 5.2 (2H, s), 5.7 (2H, s), 5.9 (2H, s), 6.7-7.9 (12H, m), 8.1 (1H, s)

2-amino-7-(2-naphthylmethyl)-3-(4-nitrobenzyl)-3,7-dihydro-6H-purin-6-one (compound 14) ¹H-NMR (400 Mhz, DMSOd₆), ppm: 5.45 (2H, s), 5.65 (2H, s), 7.05 (2H, s), 7.4-8.2 (11H, m), 8.1 (1H, s).

By catalytic reduction (5%Pd-C/H₂) of the nitro compound (compound 14),2-amino-3-(4-aminobenzyl)-7-(2-naphthyl methyl)-3,7-dihydro-6H-purin-6-one (compound 15) was obtained (85% yield). ¹H-NMR (400 Mhz, DMSOd₆), ppm: 4.95 (2H, s), 5.1 (2H, s), 5.6 (2H, s), 6.4 (2H, d), 6.8 (2H, s), 7.0 (2H, d), 7.4-7.6 (3H, m), 7.7-7.9 (4H, m), 8.1 (1H, s).

Compound 15 was also prepared as the hydrochloride salt.

2-amino-3-(3,4-difluorobenzyl)-7-(2-naphthylmethyl)-3,7-dihydro-6H-purin-6-one (compound 16). ¹H-NMR (400 Mhz, DMSOd₆), ppm: 5.2 (2H, s), 5.7 (2H, s), 7.0 (3H, m), 7.3-7.6 (5H, m), 7.8-7.9 (4H, m), 8.1 (1H, s).

4-{[2-amino-7-(2-naphthylmethyl)-6-oxo-6,7-dihydro-3H-purin-3-yl]methyl}benzonitrile (compound 17). ¹H-NMR (400 Mhz, DMSOd₆), ppm: 5.4 (2H, s), 5.7 (2H, s), 7.0 (2H, s), 7.35 (2H, d), 7.4-7.6 (3H, m), 7.7-7.9 (6H, m), 8.1 (1H, s).

By reaction of compound 17 with sodium azide and ammonium chloride in DMF at 120° C. 2-amino-7-(2-naphthylmethyl)-3-[4-(1H-tetraazol-5-yl)benzyl]-3,7-dihydro-6H-purin-6-one (compound 27) was obtained in 45% yield as a white solid. ¹H-NMR (400 Mhz, DMSOd₆), ppm: 5.35 (2H, s), 5.7 (2H, s), 7.1 (2H, s), 7.25 (2H, d), 7.3-7.75 (9H, m), 8.1 (1H, s).

2-amino-3-[4-(1H-imidazol-1-yl)benzyl]-7-(2-naphthylmethyl)-3,7-dihydro-6H-purin-6-one (compound 18). ¹H-NMR (400 Mhz, DMSOd₆), ppm: 5.3 (2H, s), 5.7 (2H, s), 7.0 (2H, s), 7.1 (1H, s), 7.3-7.9 (12H, m), 8.1 (1H, s), 8.2 (1H, m)

Compound 18 was also prepared as the bis-hydrochloride salt.

2-{[2-amino-7-(2-naphthylmethyl)-6-oxo-6,7-dihydro-3H-purin-3-yl]methyl}anthra-9,10-quinone (compound 19). ¹H-NMR (400 Mhz, DMSOd₆), ppm: 5.5 (2H, s), 5.7 (2H, s), 7.10 (2H, s), 7.40-8.25 (15H, m).

methyl 4-{[2-amino-7-(2-naphthylmethyl)-6-oxo-6,7-dihydro-3H-purin-3-yl]methyl}benzoate (compound 20). ¹H-NMR (400 Mhz, DMSOd₆), ppm: 3.8 (3H, s), 5.35 (2H, s), 5.7 (2H, s), 6.95 (2H, s), 7.15-7.9 (11H, m), 8.1 (1H, s).

By basic hydrolysis (2N NaOH in DMF) of compound 20, 4-{[2-amino-7-(2-naphthylmethyl)-6-oxo-6,7-dihydro-3H-purin-3-yl]methyl}benzoic acid (compound 21) has been isolated in 78% yield. ¹H-NMR (400 Mhz, DMSOd₆), ppm: 5.4 (2H, s), 5.65 (2H, s), 6.9 (2H, s), 7.15-7.9 (11H, m), 8.05 (1H, s).

2-amino-3-(3,4-dibenzyloxybenzyl)-7-(2-naphthylmethyl)-3,7, dihydro-6H-purin-6-one was prepared. ¹H-NMR (400 Mhz, DMSOd₆), ppm: 4.95 (2H, s), 5.05 (2H, s), 5.15 (2H, s), 5.7 (2H, s), 6.75 (1H, d), 6.85 (2H, s), 6.95 (1H, d), 7.05 (1H, s), 7.2-7.9 (17H, m), 8.1 (1H, s).

This compound treated with hydrogen in the presence of Pd on carbon gave 2-amino-3-(3,4-dihydroxybenzyl)-7-(2-naphthylmethyl)-3,7, dihydro-6H-purin-6-one (compound 22) in 68% yield. ¹H-NMR (400 Mhz, DMSOd₆), ppm: 5.45 (2H, s), 5.6 (2H, s), 6.65 (1H, d), 7.05 (1H, d), 7.3-8.0 (10H, m), 8.1 (1H, s).

2-amino-7-methyl-3-(2-naphthylmethyl)-3,7-dihydro-6H-purin-6-one (compound 23) was prepared , yield: 45%. ¹H-NMR (400 Mhz, DMSOd₆), ppm: 4.05 (3H, s), 5.6 (2H, s), 6.8-8.1 (9H, m), 8.0 (1H, s).

Starting from 7-(-methoxycarbonyl)benzyl guanine, methyl 4-{[2-amino-3-(2-naphthylmethyl)-6-oxo-3,6-dihydro-7H-purin-7-yl]methyl}benzoate (compound 26), was obtained, yield 32%. ¹H-NMR (400 Mhz, DMSOd₆), ppm: 3.8 (3H, s), 5.45 (2H, s), 5.6 (2H, s), 7.0 (2H, s), 7.3-7.9 (11H, m), 8.05 (1H, s).

By basic hydrolysis (2N NaOH in DMF) of compound 26, 4-{[2-amino-3-(2-naphthylmethyl)-6-oxo-3,6-dihydro-7H-purin-7-yl]methyl}benzoic acid (compound 32) has been isolated in 82% yield. ¹H-NMR (400 Mhz, DMSOd₆), ppm: 5.40 (2H, s), 5.65 (2H, s), 6.95 (2H, s), 7.2-7.9 (11H, m), 8.0 (1H, s).

4-({2-amino-3-[4-(methoxycarbonyl)benzyl]-6-oxo-3,6-dihydro-7H-purin-7-yl}methyl) benzoate (compound 28), yield 21%. ¹H-NMR (400 Mhz, DMSOd₆), ppm: 3.8 (6H, two s), 5.4 (2H, s), 5.6 (2H, s), 7.0 (2H, s), 7.3-7.9 (8H, m), 8.1 (1H, s).

Methyl 3-{[2-amino-7-(2-naphthylmethyl)-6-oxo-6,7-dihydro-3H-purin-3-yl]methyl}benzoate (compound 29), yield 26%. ¹H-NMR (400 Mhz, DMSOd₆), ppm: 3.8 (3H, s), 5.35 (2H, s), 5.7 (2H, s), 7.0 (2H, s), 7.3-7.9 (11H, m), 8.1 (1H, s).

By basic hydrolysis (2N NaOH in DMF) of compound 29, 3-{[2-amino-7-(2-naphthylmethyl)-6-oxo-6,7-dihydro-3H-purin-3-yl]methyl}benzoic acid (compound 30) has been isolated in 74% yield. ¹H-NMR (400 Mhz, DMSOd₆), ppm: 5.35(2H, s), 5.75 (2H, s), 6.9 (2H, s), 7.2-7.8 (11H, m), 8.15 (1H, s).

Methyl 2-{[2-amino-7-(2-naphthylmethyl)-6-oxo-6,7-dihydro-3H-purin-3-yl]methyl}benzoate (compound 31), yield 34%. ¹H-NMR (400 Mhz, DMSOd₆), ppm: 3.9 (3H, s), 5.6 (2H, s), 5.7 (2H, s), 6.9 (2H, s), 7.3-7.9 (11H, m), 8.05 (1H, s).

### EXAMPLE 5

Compound 24

To a suspension of 3-methyl guanine (0.1 g, 0.5 mmol) in anhydrous DMF (2 mL), 60%NaH (0.024 g, 0.6 mmol) is added and the mixture is stirred at room temperature for 2 h. A solution of 2-naphthylmethyl bromide (0.13 g, 0.56 mmols) in anh. DMF (1 mL) is added and the reaction mixture is stirred for 3 h at room temperature. After solvent evaporation under reduced pressure the crude reaction product is purified by flash chromatography (eluant dichloromethane/methanol 20:1) to yield 2-amino-3-methyl-7-(2-naphthymethyl)-3,7-dihydro-6H-purin-6-one as a white solid. Yield: 52%.

¹H-NMR (400 Mhz, DMSOd₆), ppm: 3.5 (3H, s), 5.7 (2H, s), 6.9 (2H,s), 7.4-7.9, (7H, m), 8.1 (1H, s).

Analogously 2-amino-7-([1,1′-biphenyl]-4-ylmethyl)-3-methyl-3,7-dihydro-6-purin-6-one (compound 25) has been prepared. Yield: 46%. ¹H-NMR (400 Mhz, DMSOd₆), ppm: 3.5 (3H, s), 5.5 (2H, s), 6.9 (2H, s), 7.3-7.6 (9H, m), 8.1 (1H, s).

### EXAMPLE 6

Intramuscular Injection of 50 mg/ml

A pharmaceutical injectable composition can be manufactured by dissolving 50 g of 3,7-bis(2-naphthylmethyl)-3,7-dihydro-6-H-purin-6-one (compound 1) in sterile propylene glycol (1000 ml) and sealed in 1-5 ml ampoules.

1. A compound which is a disubstituted purine of formula (I) 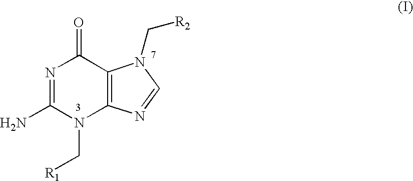
wherein
R₁ and R₂ represent each independently:
a) hydrogen;
b) phenyl unsubstituted or substituted by from 1 to 3 substituents chosen from a halogen, C₁-C₆ alkyl, C₁-C₆ alkoxy, hydroxy, carboxy, sulfo, cyano, nitro, amino, C₁-C₆ dialkylamino, a C₁-C₆ tetraalkylammonium halide, C₁-C₄ acylamino, (C₁-C₆ alkoxy)carbonyl, carbamoyl, (C₁-C₆ alkyl)carbamoyl, (C₁-C₆ dialkyl) carbamoyl, phenylcarbamoyl, guanidino, (C₁-C₆ alkyl) sulfonylamino, phenylsulfonylamino, (C₁-C₆ alkyl)aminosulfonyl, phenylaminosulfonyl, and C₅-C₇ cycloalkyl;
c) a group of formula 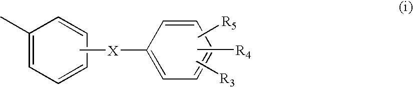
X is a bond, O or (CH₂)m wherein m is an integer from 1 to 6; R₃, R₄ and R₅ are, at the same time, hydrogen or R₃, R₄ and R₅ are chosen independently from hydrogen, a halogen, C₁-C₄ alkoxy, hydroxy, cyano, nitro, C₁-C₆ alkyl, halo C₁-C₆ alkyl, carboxy, sulfo, (C₁-C₆ alkoxy)carbonyl, amino and C₁-C₄ dialkylamino;
d) a monocyclic heteroaryl chosen from imidazolyl, pyrazolyl, oxadiazolyl, pyrrolyl, furanyl, thiadiazolyl, oxazolyl, thiazolyl, tetrazolyl, piperazinyl, N-alkyl piperazinyl, triazinyl, morpholinyl, pyridinyl, pyrimidinyl, pyrrolidinyl and piperidinyl;
e) a fused bicycle carbocyclic residue chosen from 1-naphthyl, 2-naphthyl and dihydronaphthalenyl;
f) a fused tricycle residue chosen from anthraquinonyl, phenothiazinyln, acridinyl and fluorenoyl;
g) a fused benzoheterocyclic residue chosen from benzodioxinyl, benzodioxolyl, benzofuranyl, benzothiazolyl, a benzothiazolium halide, benzothiophenyl, benzoimidazolyl, a benzoimidazolium halide, benzoxazolyl, a benzoxazolium halide, benzoxadiazolyl, quinolinyl, isoquinolinyl and quinazolinyl;
h) a phenylheterocycle residue chosen from phenylimidazolyl, phenylpyrazolyl, phenyloxadiazolyl, phenylpyrrolyl, phenylfuranyl, phenylthiadiazolyl, phenyloxazolyl, phenylthiazolyl, phenyltetrazolyl, phenylpiperazinyl, phenyl-N-alkyl piperazinyl, phenyltriazinyl, phenylmorpholinyl, phenylpyridinyl, phenylpyrimidinyl, phenylpyrrolidinyl and phenylpiperidinyl; provided that R₁ and R₂ are not at the same time hydrogen, and when R₁ is hydrogen R₂ is not unsubstituted phenyl; or a pharmaceutically acceptable salt thereof.

2. A compound according to claim 1 wherein R₁ and R₂ represent each independently:
b′) phenyl unsubstituted or substituted by from 1 to 3 substituents chosen from a halogen, C₁-C₄ alkyl, C₁-C₄ alkoxy, hydroxy, carboxy, cyano, nitro, amino, and (C₁-C₄ alkoxy)carbonyl;
c′) a group of formula (ii) or (iii) 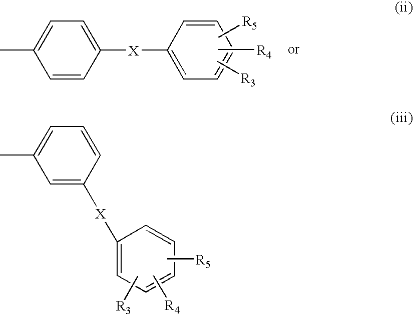
wherein
X is a bond, O or (CH₂)ₘ wherein m is an integer from 1 to 4;
R₃, R₄ and R₅ are, at the same time, hydrogen or R₃, R₄ and R₅ are chosen independently from: hydrogen, a halogen and haloC₁-C₄ alkyl;
e′) a fused bicycle carbocyclic residue chosen from 1-naphthyl and 2-naphthyl;
f′) anthraquinonyl;
g′) a fused benzoheterocyclic residue chosen from quinolinyl, benzodioxolyl and benzoxadiazolyl;
h′) a phenyl heterocycle residue chosen from phenylimidazolyl, phenyltetrazolyl, phenylpyridyl and phenyl-N-alkyl-piperazinyl); and the pharmaceutically acceptable salts thereof.

3. A compound selected from:
2-amino-3,7-bis (2-naphthylmethyl)-3,7-dihydro-6H-purin-6-one (compound 1);
2-amino-3,7-bis- [(1,1′-biphenyl)-4-ylmethyl]-3,7-dihydro-6H-purin-6-one (compound 2);
2-amino-3,7-bis{[4′-(chloromethyl) [1,1′-biphenyl]-4-yl]methyl}-3,7-dihydro-6H-purin-6-one (compound 3);
2-amino-3,7-bis(3,4-dichlorobenzyl)-3,7-dihydro-6H-purin-6-one (compound 4);
2-amino-3-([1,1′-biphenyl]-4-ylmethyl)-7-(2-naphthylmethyl)-3,7-dihydro-6H-purin-6-one (compound 5);
2-amino-3-(2-naphthylmethyl)-7-([1,1′-biphenyl]-4-ylmethyl)-3,7-dihydro-6H-purin-6-one (compound 6);
2-amino-3 (3,4-dichlorobenzyl)-7-(2-naphthylmethyl)-3,7-dihydro-6H-purin-6-one (compound 7);
2-amino-3-(3-phenoxybenzyl)-7-(2-naphthylmethyl)-3,7-dihydro-6H-purin-6-one (compound 8);
2-amino-3,7-bis (4-nitrobenzyl)-3,7-dihydro-6H-purin-6-one (compound 9);
2-amino-3,7-dibenzyl-3,7-dihydro-6H-purin-6-one (compound 10);
2-amino-3-(2-quinolinylmethyl)-7-(2-naphthylmethyl)-3,7-dihydro-6H-purin-6-one (compound 11);
2-amino-3-(2,1,3-benzoxadiazol-5-ylmethyl)-7-(2-naphthylmethyl)-3,7-dihydro-6H-purin-6-one (compound 12);
2-amino-3-(1,3-benzodioxol-5-ylmethyl)-7-(2-naphthylmethyl)-3,7-dihydro-6H-purin-6-one (compound 13);
2-amino-3-(4-nitrobenzyl)-7-(2-naphthylmethyl)-3,7-dihydro-6H-purin-6-one (compound 14);
2-amino-3-(4-aminobenzyl)-7-(2-naphthylmethyl)-3,7-dihydro-6H-purin-6-one (compound 15);
2-amino-3-(3,4-difluorobenzyl)-7-(2-naphthylmethyl)-3,7-dihydro-6H-purin-6-one (compound 16);
4-{[2-amino-7-(2-naphthylmethyl)-6-oxo-6,7-dihydro-3H-purin-3-yl]methyl}benzonitrile (compound 17);
2-amino-3-[4-(1H-imidazol-1-yl)benzyl]-7-(2-naphthylmethyl)-3,7-dihydro-6H-purin-6-one (compound 18);
2-{[2-amino-7-(2-naphthylmethyl)-6-oxo-6,7-dihydro-3H-purin-3-yl]methyl}anthra-9,10-quinone (compound 19);
methyl 4-{[2-amino-7-(2-naphthylmethyl)-6-oxo-6,7-dihydro-3H-purin-3-yl]methyl}benzoate (compound 20);
4-{[2-amino-7-(2-naphthylmethyl)-6-oxo-6,7-dihydro-3H-purin-3-yl]methyl}benzoic acid (compound 21);
2-amino-3-(3,4-dihydroxybenzyl)-7-(2-naphthylmethyl)-3,7-dihydro-6H-purin-6-one (compound 22);
2-amino-7-methyl-3-(2-naphthylmethyl)-3,7-dihydro-6H-purin-6-one (compound 23);
2-amino-3-methyl-7-(2-naphthylmethyl)-3,7-dihydro-6H-purin-6-one (compound 24);
2-amino-3-methyl-7-([1,1′-biphenyl]-4-ylmethyl)-3,7-dihydro-6H-purin-6-one (compound 25);
Methyl 4-{[2-amino-3-(2-naphthylmethyl)-6-oxo-3,6-dihydro-7H-purin-7-yl]methyl}benzoate (compound 26);
2-amino-7-(2-naphthylmethyl)-3-[4-(1H-tetraazol-5-yl)benzyl]-3,7-dihydro-6H-purin-6-one (compound 27);
Methyl 4-({2-amino-3-[4-(methoxycarbonyl)benzyl]-6-oxo-3,6-dihydro-7H-purin-7-yl}methyl) benzoate (compound 28);
Methyl 3-{[2-amino-7-(2-naphthylmethyl)-6-oxo-6,7-dihydro-3H-purin-3-yl]methyl}benzoate (compound 29);
3-{[2-amino-7-(2-naphthylmethyl)-6-oxo-6,7-dihydro-3H-purin-3-yl]methyl}benzoic acid (compound 30);
Methyl 2-{[2-amino-7-(2-naphthylmethyl)-6-oxo-6,7-dihydro-3H-purin-3-yl]methyl}benzoate (compound 31);
4-{[2-amino-3-(2-naphthylmethyl)-6-oxo-3,6-dihydro-7H-purin-7-yl]methyl}benzoic acid (compound 32) and the pharmaceutically acceptable salts thereof.

4. A process for the preparation of a compound as defined in claim 1 which comprises:
the reaction of a compound of formula (IV) 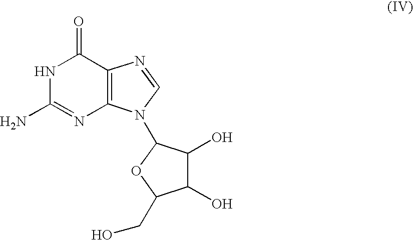
with a compound of formula (III) 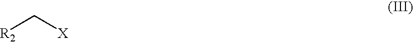
wherein X is a suitable leaving group and R₂ is as defined in claim 1; to obtain a compound of formula (V) 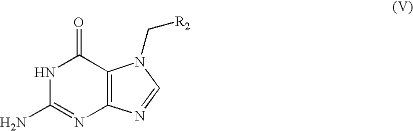
wherein R₂ is as defined above; the reaction of a compound of formula (V) with a compound of formula (VI) 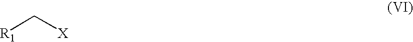
wherein X is a suitable leaving group and R₁═R₂ or wherein R₁ is different from R₂ as defined above; and the optional salification of a resulting purine of formula (I) to obtain a pharmaceutically acceptable salt.

6. A compound as defined in claim 1 for use in a method of medical treatment of the human or animal body by therapy.

7. A compound as claimed in claim 6 for use as a telomerase inhibitor.

8. A compound as claimed in claim 6, for use as an antitumor agent.

9. A pharmaceutical composition comprising a pharmaceutically acceptable carrier and/or diluent and, as an active principle, a compound of formula (I) as defined in claim 1.

10. Use of a compound as defined in claim 1 in the preparation of a medicament for use as an antitumor agent.

11. Use according to claim 10 wherein the medicament is for administration in combination chemotherapy with a second anti-tumor agent.

12. A product comprising
(a) a compound as defined in claim 1, and
(b) a second anti-tumor agent for separate, simultaneous or sequential administration to a patient suffering from cancer.

13. A method for improving the therapeutic effect of a cancer therapy which comprises administering a therapeutically effective amount of a compound of formula (I) as defined in claim 1 and at least another anticancer agent.

14. A kit comprising a compound of formula (I) as defined in claim 1 and one or more anti-cancer agents for simultaneous, separate or sequential use in anticancer therapy.

15. A compound of formula (I) as defined in claim 1 for use in treating a telomerase-modulated disease.

16. A compound of formula (I) as defined in claim 1 for use in treating a cancer disease related to a deranged cancer cell growth mediated by telomerase enzyme activity.

17. A method for treating a cancer disease which comprises administering to a patient in need thereof a therapeutically effective amount of a compound of formula (I) as defined in claim 1.
